(19) United States
(12) Patent Application Publication
(10) Pub. No.: US 2005/0261837 A1
(43) Pub. Date: Nov. 24, 2005

(54) Kernel-based system and method for estimation-based equipment condition monitoring

(75) Inventors: Stephan W. Wegerich, Geneva, IL (US); Xiao Xu, Naperville, IL (US)

Correspondence Address: FITCH EVEN TABIN AND FLANNERY, 120 SOUTH LA SALLE STREET, SUITE 1600, CHICAGO, IL, 60603-3406, US

(73) Assignee: SMARTSIGNAL CORPORATION

(21) Appl. No.: 11/121,148

(22) Filed: May 3, 2005

Related U.S. Application Data
(60) Provisional application No. 60/567,582, filed on May 3, 2004.

(51) Int. Cl. G06F 19/00; G01N 33/48; G01N 33/50

(52) U.S. Cl. 702/19

(57) ABSTRACT

A system for monitoring in real-time the health of equipment or the performance of a process utilizing a universal modeling technique that generates estimates of parameters for gauging early indications of anomalies. A kernel regression model such as the Nadaraya-Watson may be used, and may be in autoassociative form. Kernel optimization is automatically provided. A support vector regression can be substituted for the kernel regression.

### BACKGROUND OF THE INVENTION

1. Field of the Invention

The present invention relates to a method and system for monitoring the operation of a piece of equipment or a process. More particularly, it relates to equipment condition and health monitoring and process performance monitoring for early fault and deviation warning, based on non-parametric modeling and state estimation using exemplary data.

2. Description of the Related Art

Condition Based Monitoring (CBM) approaches have begun to explore kernel based modeling techniques to provide earlier actionable intelligence and machine-specific fidelity. There are a number of algorithms suited for CBM applications each with their own strengths and weaknesses.

There are many approaches to Condition Based Monitoring (CBM). The techniques range from simple trending analysis, to neural networks, to complicated expert systems. Over the past ten years or so, kernel based methods have been explored as a means for CBM. In particular, the kernel-based multivariate state estimation technique (MSET) has been used for CBM in as early as 1994. The predecessor to MSET, the system state analyzer (SSA), was applied to CBM at EBR-II in as early as 1987. More recently, support vector machines (SVM) have been shown to be applicable for CBM. It has been shown that MSET, using a similarity-based kernel at it's core, can be used as a general tool for plant-wide monitoring applications in Nuclear industry. In these applications, MSET was applied in an autoassociative manner, providing monitoring capabilities for all inputs to the MSET model. The MSET models are generated by first carefully selecting exemplars (or training vectors) from a set of base-line reference data.

Kernel Regression (KR), MSET and a general form of SVR are governed by the same basic equation. This equation is simply

$$\hat{y} = {\sum }_{i = 1}^{L} {c}_{i} K ( {x}_{new} , {x}_{i} ) , \qquad ( 1 )$$

where K(x) represents a kernel function, x_{new} is an input vector, xᵢ is a training vector and cᵢ is a coefficient that weights the kernel function output given inputs xᵢ and x_{new}. In this framework, the goal is to find an estimate of a desired output y by linearly combining the set of kernel function outputs generated from the input vector and each of the L training vectors. In the broadest sense, K(x) represents a generalized inner product between two input vectors, so the estimate is a linear combination of the generalized inner products of the input vector with each of the training vectors. Even though KR, SVR and MSET can all be represented by equation (1), there exists a significant difference in the manner in which the c's are found, and it has been discovered accordingly that KR and SVR can also be used for CBM.

### SUMMARY OF THE INVENTION

The invention provides a kernel-based modeling and estimation method and apparatus for real-time monitoring of equipment or processes. In particular, the present invention can be used for equipment health monitoring, using sensor data from the monitored equipment, to provide early warning of incipient equipment problems or upset of a monitored process.

Accordingly, the estimation module of the present invention comprises a kernel-based model created in software from exemplary data from the equipment or process to be monitored. The estimation module generates sensor value estimates of what equipment or process sensors should be registering, in response to receiving a set of actual sensor readings. The estimates of the sensors readings and the actual sensor readings are differenced to produce residuals, which under normal, healthy operation should have a mean around zero. Non-zero residuals are indicative of an incipient problem with equipment health or process operation.

The invention further provides a diagnostic rules engine that allows rules to be tested against the residuals, the estimates or the actual raw sensor values. Rules can include thresholds applied to residuals. The rules may also apply to more than one parameter at a time, such that the residual exceedance fingerprint may be mapped to a known failure mode or recognized root cause. In addition, the rules may be capable of looking at residuals, estimates and actual values over successive observations, as for example looking for a certain minimum number of residual exceedances within a window of observations (called “x in y” rules). The results of rules may identify a piece of equipment as having a certain impending health problem of failure mode, or may suggest an ameliorative action.

A graphical user interface (GUI) allows a human to review a list of rules results and equipment health statuses on a computer. The GUI also may allow the human user to drill down to the residuals, estimates and actual values, and plot these to see developing trends. These values and outputs may also be made available through software to other software systems responsible for work orders, maintenance scheduling, and operations.

The kernel-based model learns normal equipment or process behavior from reference data, comprising snapshots of readings from the same sensors that are monitored. The kernel-based model is a regression model, picked from the set of a Nadaraya-Watson kernel regression and a support vector regression. Moreover, these kernel regression models are advantageously deployed as autoassociative models in which each estimated value is corresponds to an input sensor value to the model, in contrast to inferential models in which an output value is inferred from distinct input values. A form of autoassociative support vector regression is provided by multiplexing a plurality of inferential support vector regression models, wherein each model provides an estimate for one sensor parameter.

### BRIEF DESCRIPTION OF THE DRAWINGS

The novel features believed characteristic of the invention are set forth in the appended claims. The invention itself, however, as well as the preferred mode of use, further objectives and advantages thereof, is best understood by reference to the following detailed description of the embodiments in conjunction with the accompanying drawing, wherein:

FIG. 1 is a block diagram of the modules that comprise the invention.

### DETAILED DESCRIPTION OF PREFERRED EMBODIMENTS

The present invention provides an apparatus and method for monitoring the health of a piece of equipment, or the performance of a process. It can be extended to health monitoring of any instrumented system, including biological organisms, organizations, financially defined ecosystems, and the like. Generally, the invention uses exemplary data from the machine or process in question, which forms the basis of a library of exemplars for modeling purposes. Observations from sensors or other machine or process indicators (including continuous process variables such as pressures, temperatures, etc.; fault codes, error messages, control state indicators, and other discrete data items; and “feature” values derived from other data, such as frequency features from vibration signals) are processed using a data driven kernel regression technique with reference to the stored exemplars to provide estimates for parameters of the machine or process of interest. These estimated values are compared to actually measured or determined values to produce residuals, which are the differences between the estimates and actuals. These residuals are used to indicate the presence or absence of nascent faults or other disturbances to machine health or process performance.

Accuracy and robustness of the health determination is entirely contingent on the quality of the modeled estimates for the monitored machine or process. This challenge is met in the present invention by the novel use of a model based on a kernel regression of the current observation against the library of exemplars, as is explained below. This modeling method provides improved residuals for diagnostic root cause analysis and prognosis.

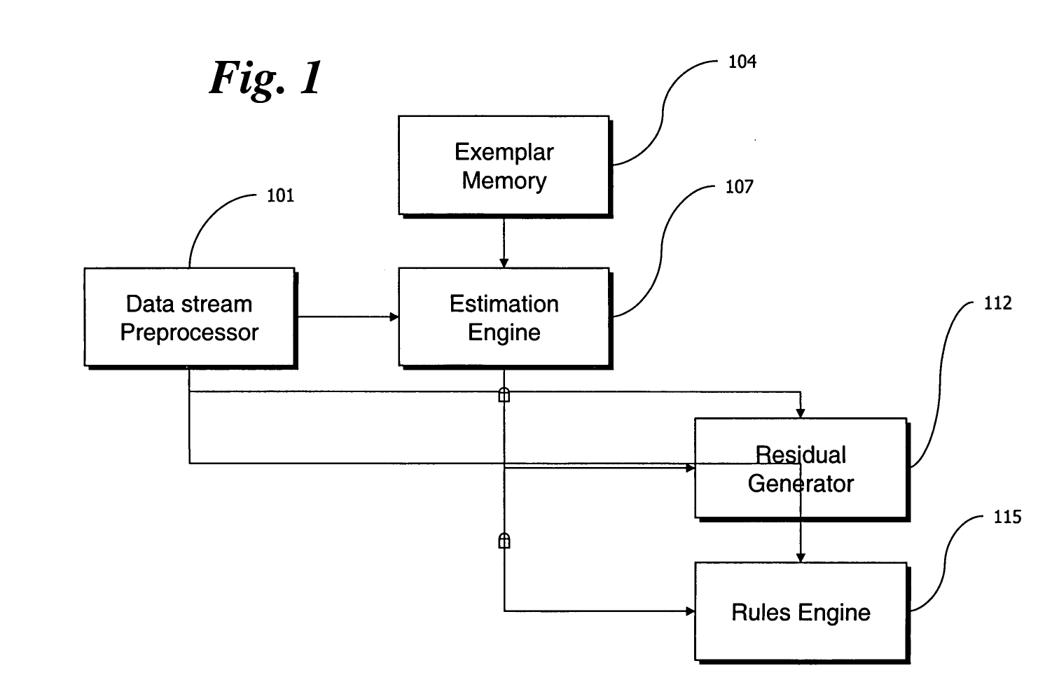

Turning to FIG. 1, the invention can generally be described as comprising a data stream preprocessor 101 disposed to receive data from sensors or from a data historian which spools sensor data from some process or system; an memory 104 module for storing the model(s) of the monitored systems in terms of the exemplars of data representative of normal or desired operational state; an estimation engine 107 responsive to the preprocessed data from preprocessor 101 for generating an estimate of an input observation using the exemplar model in memory 104; a residual generator 112 for comparing the actual data from the preprocessor 101 to the estimates of the data from the estimation engine 107, to generate residual data; and a rules-based engine 115 for executing logical tests against the residuals and/or the estimates and/or the actual data to conclude decisions with regard to system status or health.

To generate an estimate in the estimation engine 107, a kernel regression estimate can be generated. In one embodiment, the general equation used is written for a single output and multiple inputs in equation (1). The most commonly used estimator in KR is the Nadaraya-Watson estimator. Nadaraya-Watson KR weights are found by minimizing the weighted sum of squared errors shown in equation (2). The weighting is given by the kernel function output of the input and the corresponding training vector or exemplar:

$${min}_{β} {\sum }_{i = 1}^{m} {( {y}_{i} - β )}^{2} K ( {x}_{new} , {x}_{i} ) \qquad ( 2 )$$

Here, each target response value, yᵢ, corresponds to an input training vectors xᵢ. Equation (2) shows that as the kernel function output increases the contribution to the overall error increases. Therefore, the terms corresponding to the highest similarity with the input are most important to minimize. This characteristic is why KR is known a local smoothing technique. Only the terms corresponding to training vectors that are near the input contribute significantly to the overall error. If we solve equation (2) for β we get the familiar Nadaraya-Watson KR estimator shown in (3).

$$\begin{matrix}\hat{y} = \frac{{\sum }_{i = 1}^{m} {y}_{i} K ( {x}_{new} , {x}_{i} )}{{\sum }_{i = 1}^{m} K ( {x}_{new} , {x}_{i} )} & ( 3 ) \\ \hat{y} = \frac{{\sum }_{i = 1}^{L} {y}_{i} K ( {x}_{new} , {x}_{i} )}{{\sum }_{i = 1}^{L} K ( {x}_{new} , {x}_{i} )} & ( 4 )\end{matrix}$$

Now if we let
dᵢ^{out}=yᵢ and Dₒᵤₜ=└d₁^{out} d₂^{out} . . . d_{L}^{out}┘ (5)
where Dₒᵤₜ is M by L (M is the number of variables in each output vector and L is the number of training vectors) and also let
dᵢⁱⁿ=xᵢ and Dᵢₙ=└d₁ⁱⁿ d₂ⁱⁿ . . . d_{L}ⁱⁿ┘ (6)
where Dᵢₙ is N by L (N is the number of variables in each input training vector), we can re-write (4) to produce the matrix representation of the Nadaraya-Watson estimator given below.

$$\hat{y} = \frac{{\sum }_{i = 1}^{L} {d}_{i}^{out} K ( {x}_{new} , {d}_{i}^{in} )}{{\sum }_{i = 1}^{L} K ( {x}_{new} , {d}_{i}^{in} )} = \frac{{D}_{out} \cdot ( {D}_{in}^{t} ⊗ {x}_{new} )}{\sum ( {D}_{in}^{t} ⊗ {x}_{new} )} \qquad ( 7 )$$

Here, y-hat is the estimate of a parameter or set of inferential parameters made in the estimation engine 107. Hence, the estimation engine generates estimates for parameters that have been trained on, but do not make up part of the input data observation x_{new} provided by the preprocessor 101.

In an autoassociative embodiment of the estimation engine 107, the estimate contains a value for each of the input parameters in the input observation. Hence, equation (7) becomes:

$$\hat{x} = \frac{{\sum }_{i = 1}^{L} {d}_{i} K ( {x}_{new} , {d}_{i} )}{{\sum }_{i = 1}^{L} K ( {x}_{new} , {d}_{i} )} = \frac{{D}_{out} \cdot ( {D}^{t} ⊗ {x}_{new} )}{\sum ( {D}^{t} ⊗ {x}_{new} )} \qquad ( 8 )$$

where the former training vectors Dᵢₙ and Dₒᵤₜ have been combined into a single exemplar matrix, where the yᵢ and the corresponding xᵢ have been combined into single observation vectors.

A variety of kernels can be used in the invention. One well-known KR estimator kernel that can be employed is the Guassian kernel with a global bandwidth parameter h.

$$K ( {x}_{new} , {x}_{i} ) = {ⅇ}^{- \frac{{{}^{\vert {x}_{new} - {x}_{i} \vert}}^{2}}{h}} \qquad ( 9 )$$

More generally, good kernels to use for the preferred embodiment are those that meet these criteria:
symmetric with respect to the maximum
maximum when xnew=xi
non-negative

In addition, the kernel is preferably an elemental operator, meaning that the similarity of each dimension is measured and then each elemental similarity is combined (usually be averaging) to produce the final kernel function output.

Generally, finding the optimal bandwidth parameter is a matter of minimizing the error between the calculated estimate and the noise free, true output training data. Several methods can be used to optimize the bandwidth in this invention, including Akaike's Information Criterion (AIC), minimizing MSE (mean square error) based on smoothing the input, and leave-one-out Cross Validation (CV).

In AIC, a function is minimized which is equal to the sum of the log of sum of square errors and a penalty term which penalizes complexity. The penalty term is typically set to 2 times the sum of the weights divided by number of training points.

In MSE based on a smoothed input, the set of exemplars from which the model is trained is smoothed to provide an “ideal” non-noisy assumed function, which is fed back through the kernel regression model to generate estimates, which are compared to the actual smoothed function. The error is minimized to optimize the selected bandwidth for the kernel.

In leave-one-out Cross Validation, the training set of observations from which the model is learned is run back through the model to generate estimates, however at each step leaving out of the set of exemplars that make up the model the observation that is being estimated. The estimate and the actual can then be compared to provide a measure of error against which the bandwidth can be optimized.

Residuals can be generated for each observation by differencing the actual observation vector and the estimated observation vector, typically on an element-by-element basis. For inferential kernel-based models, the residual is generated by differencing the estimate of each inferred parameter with a measured value of that parameter that must be available from the data preprocessor, even though that measured value was not part of the input vector to the estimation engine. For autoassociative models, each value input to the model is estimated, and the residual is readily generated by differencing each pair.

Residuals, actual values and estimates can all be made available to the rules engine, which determines if there is evidence of a deviation in the data indicative of a change of health state for the system or process under observation. Typical rules may apply a threshold to a residual and indicate a problem if the residual exceeds the threshold. The rules may also apply to more than one parameter at a time, such that the residual exceedance fingerprint may be mapped to a predetermined ameliorative action or recognized root cause. In addition, the rules may be capable of looking at residuals, estimates and actuals over successive observations, as for example looking for a certain minimum number of residual exceedances within a window of observations (called “x in y” rules). Rules may be turned off or turned on from their processing based on conditions such as the value of certain actual data, as for example when a power parameter is monitored, and when that power parameter lies below a certain value, the rules are turned off and do not execute, so that only equipment operation above a certain level of power is monitored.

According to the invention, the results of the rules, as well as the data from residuals, estimates and actuals, can be made actionable in a variety of well-known ways, including output to a GUI interface for graphing and exception-listing, for a human to take action on. Alternatively, the results can feed into other software based systems, such as a control system for feedback control and amelioration of a faulted condition, or a work order system for issuance of a work order to explore or fix a fault.

Training data is selected from normal operating data for the system of interest. It can be downsampled by a random technique, of a more deterministic technique. For example, one way to select the exemplars that comprise the model set of exemplars D is to pick all the vectors from available historic data that contain a minimum or maximum value of any of the sensors being modeled (whether inferentially or autoassociatively) across the set of all available historic data, and then to supplement that with a sampling of randomly or otherwise chosen historic vectors, ensuring the D matrix contains at least all the observations with sensor extrema in them.

Turning to another embodiment of the present invention, a support vector regression (SVR) may be used in place of the kernel regression as described above to provide the estimate from estimation module 107. The general form for SVR is also given by equation (1). However in this case, the coefficients (cᵢ) are the solutions to a quadratic programming (QP) problem arising from the minimization of a loss function (called the ε-insensitivity loss function) with regularization constraints. The ε-insensitivity loss function is given by,
L(y, ŷ)=L(|y−ŷ|) (10)
where,

$$\vert y - \hat{y} \vert = \{ \begin{matrix}0 , & if \vert y - \hat{y} \vert \leq ɛ \\ \vert y - \hat{y} \vert - ɛ , & otherwise .\end{matrix} \qquad ( 11 )$$

This function states that the loss is equal to 0 for any discrepancies between the predicted and observed values that are less than ε. This property can have the effect of reducing over fitting of y, the estimates lie within a “tube of acceptability”. Also, it can be shown that the ε-insensitivity loss function, which is a least modulus approach as opposed to a least squares approach, provides a better solution for problems in which the noise component of y is symmetric but not necessarily Gaussian. Combining the ε-insensitivity loss function with regularization constraints, the general QP problem is formed as follows for determining the coefficients in (1) for SVR.

The coefficients cᵢ for SVR are given by cᵢ=αᵢ*−αᵢ, where αᵢ* and aᵢ are parameters that maximize

$$W = - ɛ {\sum }_{i = 1}^{L} ( {α}_{i}^{*} + {α}_{i} ) + {\sum }_{i = 1}^{L} {y}_{i} ( {α}_{i}^{*} - {α}_{i} ) - \frac{1}{2} {\sum }_{i , j = 1}^{L} ( {α}_{i}^{*} - {α}_{i} ) ( {α}_{j}^{*} - {α}_{j} ) K ( {x}_{i} , {x}_{j} ) \qquad ( 12 )$$

subject to the following constraints.

$${\sum }_{i = 1}^{L} {α}_{i}^{*} = {\sum }_{i = 1}^{L} {α}_{i} \qquad ( 13 )$$

0≦αᵢ*≦C and 0≦αᵢ≦C, i=1, . . . L (14)
The non-zero cᵢ's are defined to be the support vectors (SV) for the problem of generating the estimates, ŷᵢ, given the training example input and output pairs {xᵢ, yᵢ}.

While the above-mentioned SVR estimation method outlines an inferential estimator of y-hat in equation (1) in a univariate sense, the SVR can be extended to multiple output parameters. This can be done by building a plurality of univariate-output models using this same approach for each of the desired outputs. This means that for each output, a QP problem has to be used to solve (12) with constraints (13) and (14) each with its own resulting set of SVs. Furthermore, this can be extended to a form of autoassociative modeling (where each input is also an estimated output), by combining M such models, one for each variable, each model being an inferential univariate SVR.

Similarly, the current invention can provide an autoassociative model comprising multiple inferential kernel-regression models arranged in a similar fashion. Each kernel-regression model can be a unique inferential model that predicts one of the sensor values in the set being monitored, based on the inputs from all the other sensors. The multiple models are arranged to receive the same input vector and each model screens out of its input the variable it is predicting. The predictions are assembled from all the individual models to provide an overall estimate of all the sensors that were in the original input vector, hence an autoassociative estimate.

It should be appreciated that a wide range of changes and modifications may be made to the embodiments of the invention as described herein. Thus, it is intended that the foregoing detailed description be regarded as illustrative rather than limiting and that the following claims, including all equivalents, are intended to define the scope of the invention.

1. An apparatus for monitoring the condition of an instrumented system, comprising:
a memory for storing data exemplars characterizing normal operation of said system;
a processor-executable estimation module disposed to generate estimates of operational parameters of said system in response to receiving measurements of operational parameters, by performing an autoassociative kernel-based regression using said data exemplars and the received measurements; and
a processor-executable comparison module disposed to compare said estimates of operational parameters with corresponding said measurements of operational parameters to identify residuals indicative of system condition.

2. An apparatus as recited in claim 1, further comprising a processor-executable diagnostic module disposed to determine at least one diagnostic condition for said system on the basis of the residuals identified by said processor-executable comparison module.

3. An apparatus as recited in claim 2, wherein said processor-executable diagnostic module comprises a rule execution engine for processing said residuals with rules to determine at least one diagnostic condition.

4. An apparatus as recited in claim 2, further comprising a processor-executable annunciation module disposed to generate condition messages descriptive of diagnostic conditions determined by said diagnostic module.

5. An apparatus as recited in claim 1, wherein said estimation module performs a Nadaraya-Watson kernel regression to provide autoassociative estimates of operational parameters according to the equation:
$$\hat{x} = \frac{{\sum }_{i = 1}^{L} {d}_{i} K ( {x}_{new} , {d}_{i} )}{{\sum }_{i = 1}^{L} K ( {x}_{new} , {d}_{i} )} = \frac{D \cdot ( {D}^{t} ⊗ {x}_{new} )}{\sum ( {D}^{t} ⊗ {x}_{new} )}$$

6. An apparatus according to claim 5, wherein the kernel K is symmetric with respect to its maximum value, and produces that maximum value when comparing identical vectors.

7. An apparatus according to claim 6, wherein the kernel K is a Gaussian kernel.

8. An apparatus according to claim 1, wherein said estimation module performs a bank of inferential kernel regressions, each kernel regression predicting one of said operational parameters using at least some of the other operational parameters as input, and integrates the predictions into an autoassociative estimate of at least some of the operational parameters.

9. An apparatus according to claim 1, wherein said estimation module performs a support vector regression to provide estimates of operational parameters.

10. An apparatus according to claim 9, wherein said estimation module performs a bank of support vector regressions, each of which provides an inferential estimate of one operational parameter using at least some of the other operational parameters as input, and integrates the estimates into an autoassociative estimate of at least some of the operational parameters.

11. A method for monitoring the condition of an instrumented system, comprising the steps of:
providing a set of reference observations of operational parameters of said instrumented system;
measuring a set of operational parameters from said instrumented system;
generating estimates for at least some of the operational parameters based on a kernel-based regression of the measured set of operational parameters;
differencing the generated estimates and the measured operational parameters to produce residuals indicative of the condition of said instrumented system.

12. A method according to claim 11, further comprising the step of determining at least one diagnostic condition for said system on the basis of the residuals.

13. A method according to claim 12, wherein said step of determining at least one diagnostic condition comprises processing said residuals with rules to determine the at least one diagnostic condition.

14. A method according to claim 13, further comprising the step of generating condition messages descriptive of diagnostic conditions determined in said diagnostic condition determining step.

15. A method according to claim 11, wherein said estimate generating step comprises generating at least one autoassociative estimate of an operational parameter according to a Nadaraya-Watson kernel regression of the form:
$$\hat{x} = \frac{{\sum }_{i = 1}^{L} {d}_{i} K ( {x}_{new} , {ⅆ}_{i} )}{{\sum }_{i = 1}^{L} K ( {x}_{new} , {ⅆ}_{i} )} = \frac{D \cdot ( {D}^{t} ⊗ {x}_{new} )}{\sum ( {D}^{t} ⊗ {x}_{new} )} .$$

16. A method according to claim 15, wherein the kernel K is symmetric with respect to its maximum value, and produces that maximum value when comparing identical vectors.

17. A method according to claim 16, wherein the kernel K is a Gaussian kernel.

18. A method according to claim 11, wherein said estimate generating step comprises performing a plurality of inferential kernel regressions, each kernel regression predicting one of said operational parameters using at least some of the other operational parameters as input, and integrating the predictions into an autoassociative estimate of at least some of the operational parameters.

19. A method according to claim 11, wherein said estimate generating step comprises performing a support vector regression to provide estimates of operational parameters.

20. A method according to claim 19, wherein said estimate generating step comprises performing a plurality of of support vector regressions, each of which provides an inferential estimate of one operational parameter using at least some of the other operational parameters as input, and integrating the estimates into an autoassociative estimate of at least some of the operational parameters.
